(19) United States
(12) Patent Application Publication
(10) Pub. No.: US 2021/0153929 A1
(43) Pub. Date: May 27, 2021

(54) TREATMENT TOOL AND TREATMENT TOOL AIRTIGHT MEMBER

(71) Applicant: OLYMPUS CORPORATION, Tokyo (JP)

(72) Inventors: Takahito OSHIRO, Tokyo (JP); Yoshitaka FUJII, Atsugi-shi (JP)

(73) Assignee: OLYMPUS CORPORATION, Tokyo (JP)

(21) Appl. No.: 17/165,010

(22) Filed: Feb. 2, 2021

Related U.S. Application Data
(63) Continuation-in-part of application No. PCT/JP2018/029312, filed on Aug. 3, 2018.

(51) Int. Cl. A61B 18/14

(52) U.S. Cl. CPC A61B 18/1445; A61B 2018/00994

(57) ABSTRACT

A treatment tool includes: a handle; an elongated fixation member a proximal end side of which is fixed to the handle; a movable member that is provided coaxially with the fixation member, the movable member being configured to move with respect to the fixation member; and an airtight member which is provided between the fixation member and the movable member and in which a second contact width in contact with the movable member is smaller than a first contact width in contact with the fixation member.

### CROSS-REFERENCE TO RELATED APPLICATION

This application is a continuation-in-part of International Application No. PCT/JP2018/029312, filed on Aug. 3, 2018, the entire contents of which are incorporated herein by reference.

### BACKGROUND

### 1. Technical Field

The disclosure relates to a treatment tool and a treatment tool airtight member.

### 2. Related Art

In the related art, a treatment tool in which a unit to apply ultrasound energy (ultrasound vibration) or high-frequency energy (high-frequency current) to a body tissue is provided and which gives treatment (such as joining (or anastomosis) and dissection) to the body tissue, for example, by applying the ultrasound vibration has been known (see, for example, JP 2009-240773 A).

In JP 2009-240773 A, a treatment tool including two gripping members (first and second gripping members) that grip a body tissue is described. In JP 2009-240773 A, the first gripping member is connected to a first pipe member inserted into the treatment tool. The first pipe member is covered with the second pipe member, and moves in the second pipe member by operation by an operator. The first gripping member moves closer to or away from the other gripping member in conjunction with the movement of the first pipe member. By this movement of the first gripping member, a body tissue can be sandwiched and gripped between the first and second gripping members.

When the treatment tool is used, an abdominal cavity is widened for treatment. Pressure (hereinafter, also referred to as abdominal air pressure) is applied to the abdominal cavity to widen the abdominal cavity. Also, in the treatment tool of JP 2009-240773 A, a gap between the first pipe member and the second pipe member is sealed by an O-ring. By this O-ring, a decrease in the abdominal air pressure due to leakage of gas in the abdominal cavity from a gap between the first pipe member and the second pipe member is controlled while the first pipe member is caused to slide with respect to the second pipe member.

### SUMMARY

In some embodiments, a treatment tool includes: a handle; an elongated fixation member a proximal end side of which is fixed to the handle; a movable member that is provided coaxially with the fixation member, the movable member being configured to move with respect to the fixation member; and an airtight member which is provided between the fixation member and the movable member and in which a second contact width in contact with the movable member is smaller than a first contact width in contact with the fixation member.

In some embodiments, provided is a treatment tool airtight member that is provided between a fixation member and a movable member, the treatment tool airtight member being configured to airtightly seal a gap between the fixation member and the movable member, a proximal end side of the fixation member being fixed to a handle, the movable member being provided coaxially with the fixation member and moving with respect to the fixation member. In arrangement of the treatment tool airtight member between the fixation member and the movable member, a second contact width in contact with the movable member is smaller than a first contact width in contact with the fixation member.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a view illustrating a treatment device according to one embodiment of the disclosure;

FIG. 2 is a cross-sectional view illustrating a transducer unit in a treatment tool according to the one embodiment of the disclosure;

FIG. 3 is a cross-sectional view illustrating an internal configuration of a handle in the treatment tool according to the one embodiment of the disclosure;

FIG. 4 is a cross-sectional view illustrating a distal end configuration of the treatment tool according to the one embodiment of the disclosure;

FIG. 5 is an exploded perspective view illustrating the distal end configuration of the treatment tool according to the one embodiment of the disclosure;

FIG. 6 is a partial cross-sectional view illustrating an internal configuration of a holding unit in the treatment tool according to the one embodiment of the disclosure;

FIG. 7 is a perspective view illustrating a configuration of an airtight member included in the treatment tool according to the one embodiment of the disclosure;

FIG. 8 is a cross-sectional view illustrating a part of the configuration of the airtight member included in the treatment tool according to the one embodiment of the disclosure;

FIG. 9A is a cross-sectional view illustrating a part of the configuration of the airtight member included in the treatment tool according to the one embodiment of the disclosure;

FIG. 9B is a cross-sectional view illustrating a part of the configuration of the airtight member included in the treatment tool according to the one embodiment of the disclosure;

FIG. 10 is a cross-sectional view illustrating a part of the configuration of the airtight member included in the treatment tool according to the one embodiment of the disclosure; and

FIG. 11 is a cross-sectional view illustrating a configuration of an airtight member included in a treatment tool according to a modification example of an embodiment of the disclosure.

### DETAILED DESCRIPTION

In the following, a treatment tool and a treatment tool airtight member according to embodiments of the disclosure will be described with reference to the drawings. Note that the disclosure is not limited to these embodiments. Also, the same reference sign is assigned to identical parts in the drawings.

### Embodiment

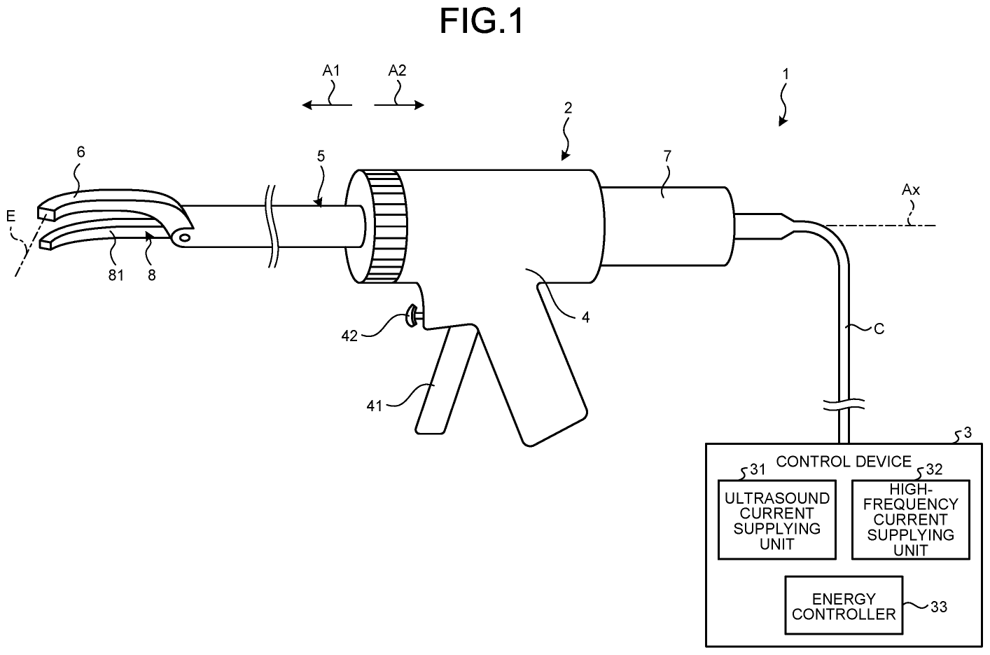

FIG. 1 is a schematic diagram illustrating a treatment device according to one embodiment of the disclosure. By applying ultrasound energy or high-frequency energy to a region to be treated (hereinafter, referred to as target region) in a body tissue, a treatment device 1 treats the target region. Here, the treatment means, for example, coagulation and incision of the target region. This treatment device 1 includes an ultrasound treatment tool 2 and a control device 3.

The ultrasound treatment tool 2 is, for example, a medical treatment tool using a bolt-clamped Langevin type transducer (BLT) to treat a target region in a state of penetrating an abdominal wall. This ultrasound treatment tool 2 includes a handle 4, a sheath 5, a jaw 6, a transducer unit 7, and an ultrasound probe 8.

The handle 4 is a part held by an operator. As illustrated in FIG. 1, an operation knob 41 and an operation button 42 are provided in this handle 4.

The sheath 5 has a cylindrical shape. Note that a central axis of the sheath 5 will be referred to as a central axis Ax in the following. Also, one side along the central axis Ax will be referred to as a distal end side A1, and the other side will be referred to as a proximal end side A2 in the following. The sheath 5 has an elongated shape extending along the central axis Ax. Then, the sheath 5 is attached to the handle 4 by insertion of a part of the proximal end side A2 into the inside of the handle 4 from the distal end side A1 of the handle 4. An internal configuration of the sheath 5 will be described later.

The jaw 6 is rotatably attached to an end portion on the distal end side A1 of the sheath 5 and grips a target region with a part on the distal end side A1 of the ultrasound probe 8. Note that an opening/closing mechanism that opens/closes the jaw 6 with respect to the part on the distal end side A1 of the ultrasound probe 8 according to operation of the operation knob 41 by the operator is provided inside the handle 4 and the sheath 5 described above.

In this jaw 6, a resin pad or a swing member (not illustrated) that swings with respect to a jaw main body is preferably attached to a surface facing the ultrasound probe 8, for example. This pad can prevent the ultrasound probe 8, which makes an ultrasound vibration, from being damaged by colliding with the jaw 6 when incision of the target region by the ultrasound vibration is completed. Also, this pad has an insulation property and can prevent a short circuit when high-frequency energy is applied between the jaw 6 and the ultrasound probe 8.

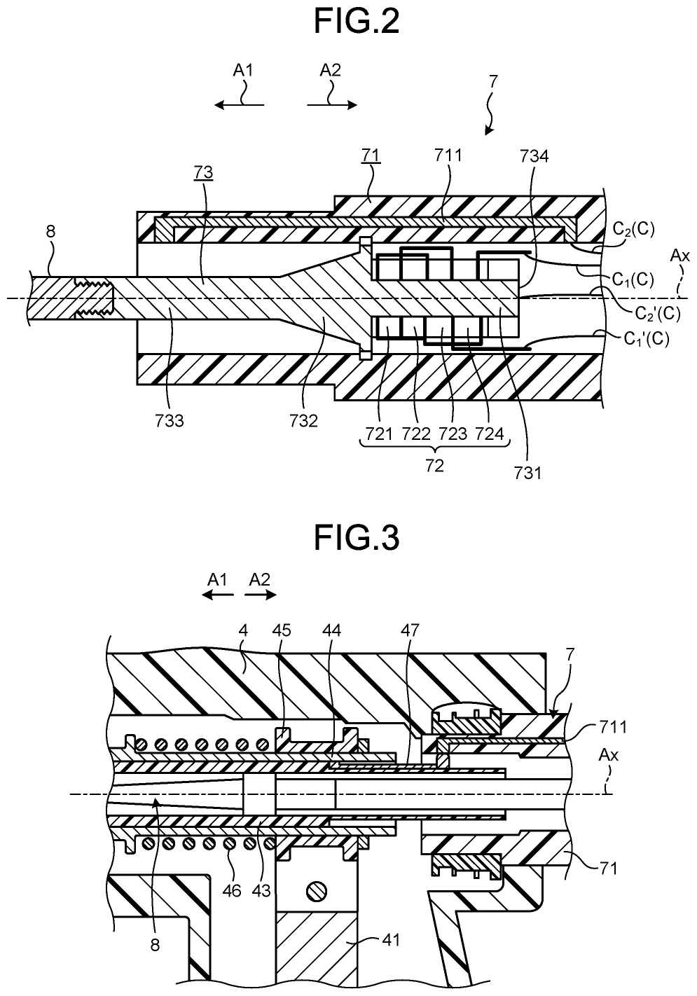

FIG. 2 is a cross-sectional view illustrating the transducer unit 7. More specifically, FIG. 2 is a cross-sectional view of the transducer unit 7 cut by a plane including the central axis Ax. As illustrated in FIG. 2, the transducer unit 7 includes a transducer case 71, an ultrasound transducer 72, and a horn 73.

The transducer case 71 extends linearly along the central axis Ax, and is attached to the handle 4 by insertion of a part thereof on the distal end side A1 into the inside of the handle 4 from the proximal end side A2 of the handle 4. Then, in a state in which the transducer case 71 is attached to the handle 4, an end portion thereof on the distal end side A1 is coupled to an end portion on the proximal end side A2 of the sheath 5.

The ultrasound transducer 72 is housed inside the transducer case 71, and generates an ultrasound vibration under the control of the control device 3. In the present embodiment, the ultrasound vibration is a longitudinal vibration that vibrates in a direction along the central axis Ax. This ultrasound transducer 72 is a BLT including a plurality of piezoelectric elements 721 to 724 laminated along the central axis Ax (see FIG. 2). Note that four piezoelectric elements 721 to 724 are provided in the present embodiment, but the number thereof is not limited to four and may be any other number.

The horn 73 is housed inside the transducer case 71, and expands amplitude of the ultrasound vibration generated by the ultrasound transducer 72. This horn 73 has an elongated shape extending linearly along the central axis Ax. Then, from the proximal end side A2 to the distal end side A1, the horn 73 includes a transducer mounting portion 731 to which the ultrasound transducer 72 is mounted, a cross-sectional area change portion 732 that has a shape, with which a cross-sectional area decreases toward the distal end side A1, and that expands the amplitude of the ultrasound vibration, and a probe mounting portion 733 to which the ultrasound probe 8 is mounted (see FIG. 2).

The ultrasound probe 8 has an elongated shape extending linearly along the central axis Ax, and is inserted into the inside of the sheath 5 in a state in which a part on the distal end side A1 protrudes outward (see FIG. 1). Also, an end portion on the proximal end side A2 of the ultrasound probe 8 is connected to the probe mounting portion 733 (see FIG. 2). On the one hand, a treatment unit 81 that grips and treats a target region with the jaw 6 is provided in an end portion on the distal end side A1 of the ultrasound probe 8. Then, the ultrasound probe 8 treats the target region by transmitting the ultrasound vibration generated by the ultrasound transducer 72 from the end portion on the proximal end side A2 to the end portion on the distal end side A1 (treatment unit 81) through the horn 73, and applying the ultrasound vibration from the treatment unit 81 to the target region.

The control device 3 is electrically connected to the ultrasound treatment tool 2 by an electric cable C (see FIG. 1), and comprehensively controls an operation of the ultrasound treatment tool 2. This control device 3 includes an ultrasound current supplying unit 31, a high-frequency current supplying unit 32, and an energy controller 33 (see FIG. 1).

Here, a pair of transducer lead wires C₁ and C₁′ included in the electric cable C are joined to the ultrasound transducer 72 (see FIG. 2).

Then, the ultrasound current supplying unit 31 supplies AC power to the ultrasound transducer 72 through the pair of transducer lead wires C₁ and C₁′ under the control of the energy controller 33. As a result, the ultrasound transducer 72 generates an ultrasound vibration.

Here, a first conductive portion 711 extending from the end portion on the proximal end side A2 to the end portion on the distal end side A1 is provided in the transducer case 71 (see FIG. 2). Also, although detailed illustration is omitted, a second conductive portion that extends from the end portion on the proximal end side A2 to the end portion on the distal end side A1 and that electrically connects the first conductive portion 711 and the jaw 6 is provided in the sheath 5. Also, a high-frequency lead wire C₂ included in the electric cable C is joined to an end portion on the proximal end side A2 of the first conductive portion 711. Furthermore, a high-frequency lead wire C₂′ included in the electric cable C is joined to an end portion (end portion 734) of the transducer mounting portion 731.

Then, under the control of the energy controller 33, the high-frequency current supplying unit 32 supplies a high frequency current between the jaw 6 and the ultrasound probe 8 through the pair of high-frequency lead wires C₂ and C₂′, the first conductive portion 711, the second conductive portion, and the horn 73. As a result, a high-frequency current flows in the target region gripped between the jaw 6 and the part on the distal end side A1 of the ultrasound probe 8. That is, the jaw 6 and the ultrasound probe 8 also function as high frequency electrodes. In other words, the ultrasound treatment tool 2 also functions as a bipolar treatment tool when the jaw 6 and the ultrasound probe 8 function as a pair of high frequency electrodes.

The energy controller 33 is, for example, a central processing unit (CPU), a field-programmable gate array (FPGA), or the like, and controls operations of the ultrasound current supplying unit 31 and the high-frequency current supplying unit 32 according to a predetermined control program in a case where the operation button 42 is pressed by the operator.

FIG. 3 is a view illustrating the internal configuration of the handle 4. A connecting tubular portion 43 formed of an insulating material (non-conductive material), and a movable tubular portion 44 provided on a side of an outer peripheral direction of the connecting tubular portion 43 are provided inside the handle 4.

The movable tubular portion 44 is formed of a conductive material and can move along a longitudinal axis Ax with respect to the transducer case 71 and the connecting tubular portion 43. A slider member 45 formed of an insulating material (non-conductive material) is provided in an outer peripheral portion of the movable tubular portion 44.

The slider member 45 can move along the longitudinal axis Ax with respect to the movable tubular portion 44. An elastic member 46 is provided between the slider member 45 and the movable tubular portion 44. The elastic member 46 includes a coil spring or the like.

Also, the operation knob 41 is attached to the slider member 45. When the operation knob 41 is opened/closed with respect to the handle 4, driving force is transmitted to the slider member 45, and the slider member 45 moves along the longitudinal axis Ax. Then, the driving force is transmitted from the slider member 45 to the movable tubular portion 44 via the elastic member 46, and the movable tubular portion 44 moves along the longitudinal axis Ax with respect to the transducer case 71 and the connecting tubular portion 43.

Also, a plate-shaped contact member 47 formed of a conductive material is fixed to the connecting tubular portion 43. In a state in which the transducer case 71 is connected to the handle 4, one end of the contact member 47 abuts on the first conductive portion 711 of the transducer case 71, and the movable tubular portion 44 movably abuts on the other end of the contact member 47. Thus, in a state in which the transducer case 71 is connected to the handle 4, the first conductive portion 711 of the transducer case 71 and the movable tubular portion 44 are electrically connected via the contact member 47. As a result, high-frequency energy is supplied (transmitted) from the high-frequency current supplying unit 32 to the movable tubular portion 44 of the sheath 5 through electric wiring 48 and the first conductive portion 711 of the transducer case 71. Note that the first conductive portion 711 of the transducer case 71 and the movable tubular portion 44 of the sheath 5 are electrically insulated from the horn 73 and the ultrasound probe 8.

The energy controller 33 controls an output state of ultrasound energy from the ultrasound current supplying unit 31 and an output state of high-frequency energy from the high-frequency current supplying unit 32 on the basis of an input of energy operation triggered by pressing on the operation button 42. A switch (not illustrated) is provided inside the handle 4. When the operation button 42 is pressed and the energy operation is input, the switch is closed. The switch is electrically connected to the energy controller 33. When the switch is closed, an electric signal is transmitted to the energy controller 33, and the input of the energy operation is detected. When the input of the energy operation is detected, ultrasound energy is output from the ultrasound current supplying unit 31, and high-frequency energy is output from the high-frequency current supplying unit 32.

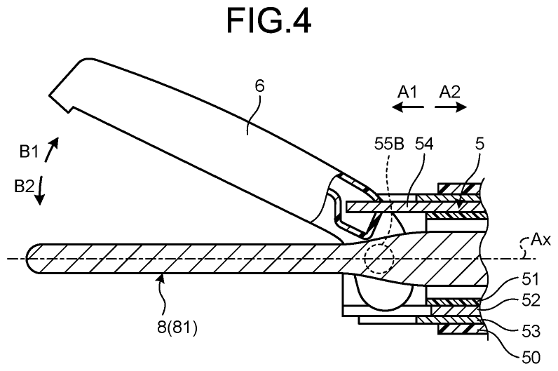

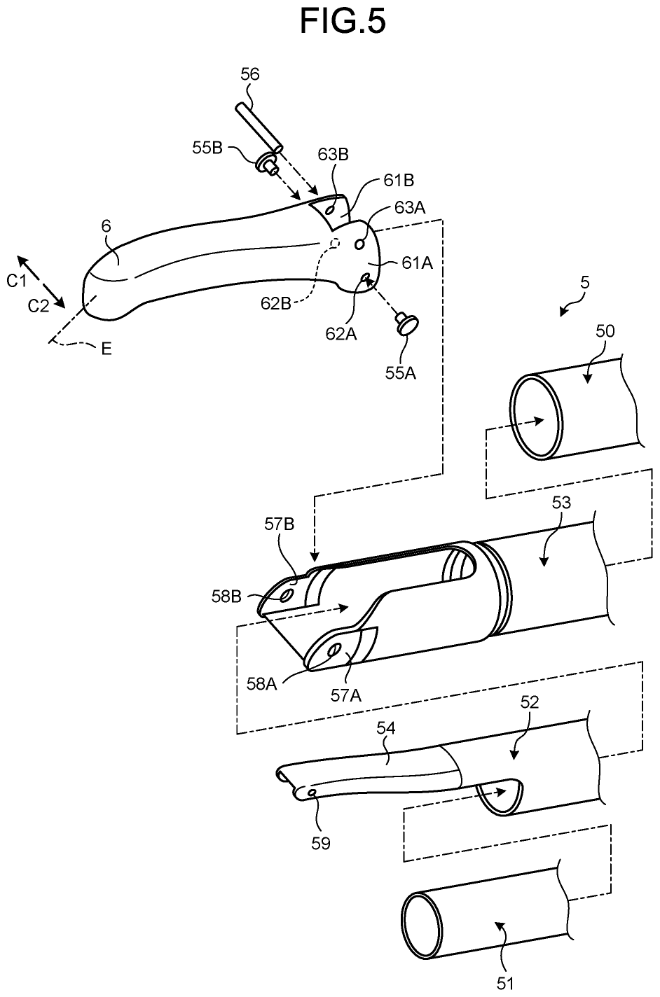

FIG. 4 is a cross-sectional view illustrating a distal end configuration of the treatment tool according to the one embodiment of the disclosure. FIG. 5 is an exploded perspective view illustrating the distal end configuration of the treatment tool according to the one embodiment of the disclosure. The jaw 6 extends along an extension axis (jaw shaft) E (see FIG. 1) from a proximal end direction to a distal end direction. The extension axis E is a central axis of the jaw 6. In a case where the jaw 6 is closed with respect to the ultrasound probe 8, the extension axis E of the jaw 6 is substantially parallel to the longitudinal axis Ax. One of directions perpendicular to the longitudinal axis Ax and the extension axis E is an opening direction of the jaw 6 (direction of an arrow B1 in FIG. 4), and a direction opposite to the opening direction is a closing direction of the jaw 6 (direction of an arrow B2 in FIG. 4). Also, two directions perpendicular to the extension axis E (longitudinal axis Ax) and perpendicular to the opening/closing direction of the jaw 6 are width directions. One of the width directions is a first width direction (direction of an arrow C1 in FIG. 5), and the other of the width directions is a second width direction (direction of an arrow C2 in FIG. 5). Note that FIG. 4 is illustrated in a cross section perpendicular to the width directions. Also, FIG. 4 is a view illustrating a state in which the jaw 6 is opened with respect to the ultrasound probe 8.

A pair of jaw protrusion pieces 61A and 61B are provided in the proximal end portion of the jaw 6 (see FIG. 5). The jaw protrusion piece 61A is placed on a side of the first width direction (arrow C1 side) of the jaw protrusion piece 61B. A space is formed between the jaw protrusion piece 61A and the jaw protrusion piece 61B. A through hole 62A penetrating in the width directions is formed in the jaw protrusion piece 61A. Also, a through hole 62B penetrating the jaw protrusion piece 61B in the width directions is formed in the jaw protrusion piece 61B.

Also, a connection hole 63A penetrating the jaw protrusion piece 61A in the width directions is formed in the jaw protrusion piece 61A. Also, a connection hole 63B penetrating the jaw protrusion piece 61B in the width directions is formed in the jaw protrusion piece 61B.

At the distal end of the ultrasound probe 8, the treatment unit 81 (longitudinal axis Ax) is curved in the first width direction (see FIG. 1). With the curve of the distal end of the ultrasound probe 8 (treatment unit 81), visibility for an operator is improved during treatment. Also, in the jaw 6, the jaw 6 (extension axis E) is curved in the first width direction in a manner corresponding to a curve mode of the ultrasound probe 8. Since the jaw 6 is also curved, the jaw 6 extends in a state of facing the ultrasound probe 8 (treatment unit 81).

Subsequently, the sheath 5 will be described with reference to FIG. 5.

The sheath 5 includes an inner tube 51 into which the ultrasound probe 8 is inserted, a movable pipe 52 provided on an outer peripheral side of the inner tube 51, an outer pipe 53 provided on the outer peripheral side of the movable pipe 52, and an outer tube 50 provided on the outer peripheral side of the outer pipe 53. The movable pipe 52 and the outer pipe 53 are provided coaxially. In the present specification, the movable pipe 52 corresponds to a movable member, and the outer pipe 53 corresponds to a fixation member.

The outer tube 50 and the inner tube 51 are formed of an insulating material (non-conductive material).

The movable pipe 52 and the outer pipe 53 are formed of a conductive material.

A movable protrusion 54 is formed in a distal end portion of the movable pipe 52. The movable protrusion 54 is placed in a space between the jaw protrusion piece 61A and the jaw protrusion piece 61B in the width directions. Also, a through hole 59 that penetrates the movable protrusion 54 in the width directions is formed in the movable protrusion 54. A distal end portion of the movable pipe 52 is connected to the jaw 6 via a connection pin 56 that is a connection member. The connection pin 56 is inserted into the connection hole 63A of the jaw protrusion piece 61A, the through hole 59 of the movable protrusion 54, and the connection hole 63B of the jaw protrusion piece 61B. The connection pin 56 is in contact with the movable pipe 52 at the movable protrusion 54, and is also in contact with the jaw 6 at the jaw protrusion piece 61A and the jaw protrusion piece 61B.

A proximal end portion of the movable pipe 52 is coupled to an end portion on the distal end side A1 of the movable tubular portion 44. When driving force is transmitted to the movable pipe 52 by the closing operation of the operation knob 41 with respect to the handle 4, the movable pipe 52 moves together with the movable tubular portion 44 along the longitudinal axis Ax with respect to the inner tube 51, the outer pipe 53, and the outer tube 50. As the movable tubular portion 44 and the movable pipe 52 move along the longitudinal axis Ax, the jaw 6 performs a closing operation or an opening operation with respect to the ultrasound probe 8 (treatment unit 81).

A pair of sheath protrusion pieces 57A and 57B are provided in the distal end portion of the outer pipe 53. A through hole 58A penetrating in the width directions is formed in the sheath protrusion piece 57A. The sheath protrusion piece 57A abuts on the jaw protrusion piece 61A from a side of the first width direction. Also, a through hole 58B penetrating in the width directions is formed in the sheath protrusion piece 57B. The sheath protrusion piece 57B abuts on the jaw protrusion piece 61B from a side of the second width direction. An end portion on the proximal end side A2 of the outer pipe 53 is fixed to the handle 4.

The jaw 6 is attached to the distal end portion of the outer pipe 53 of the sheath 5 with fulcrum pins 55A and 55B. The jaw 6 rotates about a rotation axis coaxial with a central axis of each of the fulcrum pins 55A and 55B. This rotation axis is substantially parallel to the width directions (C1 and C2).

In a state in which the jaw 6 is attached to the sheath 5, the fulcrum pin 55A is inserted into the through hole 58A in the sheath protrusion piece 57A and the through hole 62A in the jaw protrusion piece 61A from the side of the first width direction, and the fulcrum pin 55B is inserted into the through hole 58B in the sheath protrusion piece 57B and the through hole 62B in the jaw protrusion piece 61B from the side of the second width direction.

Here, the high-frequency energy transmitted from the high-frequency current supplying unit 32 to the movable tubular portion 44 is transmitted to the movable pipe 52 via a fuse pin (not illustrated). In the present embodiment, a high-frequency transmission portion (jaw-side high-frequency transmission portion) is formed by the movable tubular portion 44 and the movable pipe 52 of the sheath 5. Then, the ultrasound probe 8 is inserted into the high-frequency transmission portion (movable tubular portion 44 and movable pipe 52). That is, the movable tubular portion 44 and the movable pipe 52 become sheath conductive portions capable of transmitting a high-frequency current in the sheath 5. Note that the movable pipe 52 that is the high-frequency transmission portion is electrically insulated from the ultrasound probe 8.

The high-frequency energy transmitted to the movable pipe 52 (high-frequency transmission portion) is transmitted to the jaw 6 via the connection pin 56. Thus, a jaw-side electric path is formed from the high-frequency current supplying unit 32 to the jaw 6 via the electric wiring 48, the first conductive portion 711 of the transducer case 71, the movable tubular portion 44, and the movable pipe 52. High-frequency energy (high-frequency power) is transmitted (supplied) from the high-frequency current supplying unit 32 to the jaw 6 by the jaw-side electric path.

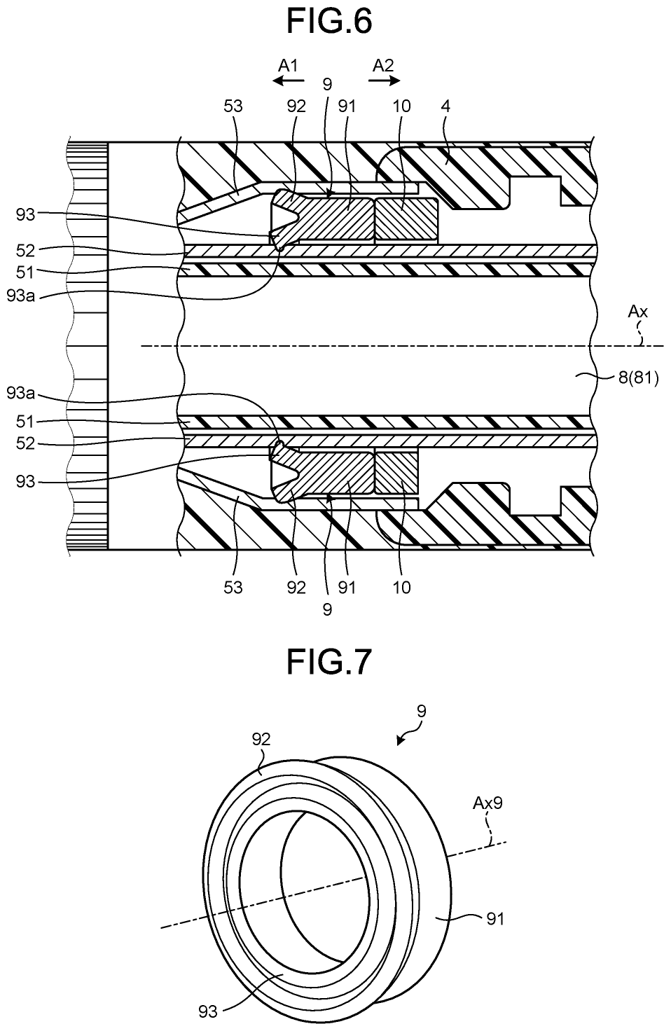

Subsequently, a configuration on the proximal end side of the sheath 5 will be described with reference to FIG. 6 to FIG. 8. FIG. 6 is a partial cross-sectional view illustrating an internal configuration of a holding unit in the treatment tool according to the one embodiment of the disclosure. An inner diameter of a proximal end portion of the outer pipe 53 is larger than inner diameters of a distal end portion and a central portion thereof. An airtight member 9 is arranged between the proximal end portion of the outer pipe 53 and the movable pipe 52, and a gap between the outer pipe 53 and the movable pipe 52 is sealed. That is, the airtight member 9 seals the proximal end side of the movable pipe 52 and the outer pipe 53. Also, the airtight member 9 abuts on a stopper 10, and movement thereof to the proximal end side A2 is restricted. The stopper 10 is fixed to the movable pipe 52 and/or the outer pipe 53.

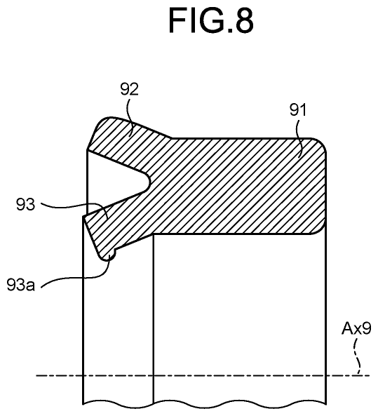

FIG. 7 is a perspective view illustrating a configuration of the airtight member included in the treatment tool according to the one embodiment of the disclosure. FIG. 8 is a cross-sectional view illustrating a part of the configuration of the airtight member included in the treatment tool according to the one embodiment of the disclosure. FIG. 8 is a cross section cut by a plane that is parallel to a central axis Ax9 of the airtight member 9 and that includes the central axis Ax9. The airtight member 9 is in contact with the outer pipe 53 on an outer peripheral side and is in contact with the movable pipe 52 on an inner peripheral side (see FIG. 6).

The airtight member 9 has a cylindrical main body portion 91, a first arm portion 92 extending from the main body portion 91 in a manner of being inclined in a direction of becoming away from the central axis Ax9, and a second arm portion 93 extending from the main body portion 91 in a manner of being inclined in a direction of becoming closer to the central axis Ax9. The airtight member 9 is formed of an elastically deformable material such as rubber or resin. The central axis Ax9 corresponds to a central axis of a cylindrical shape of the main body portion 91.

The first arm portion 92 and the second arm portion 93 extend from an end portion on the same side between end portions of the central axis Ax9 of the main body portion 91.

The first arm portion 92 extends from one end in a direction of the central axis Ax9 of the main body portion 91 while expanding a diameter. A distal end of the first arm portion 92 is placed on the outermost peripheral side of the airtight member 9. Here, a natural state means a state in which a load other than gravity is not applied from the outside.

The second arm portion 93 extends from one end in the direction of the central axis Ax9 of the main body portion 91 while reducing a diameter. A protrusion portion 93a is formed at a distal end on an inner peripheral side of the second arm portion 93. The protrusion portion 93a is placed on the innermost peripheral side of the airtight member 9 in the natural state.

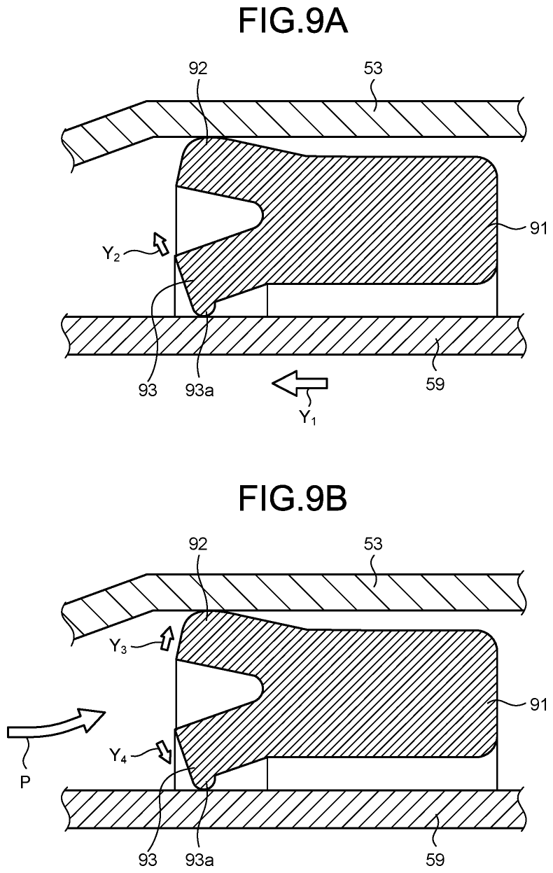

When the airtight member 9 is attached to the movable pipe and the outer pipe 53 (see FIG. 6), the first arm portion 92 is pressed against an inner peripheral surface of the outer pipe 53, and the protrusion portion 93a of the second arm portion 93 is pressed against an outer peripheral surface of the movable pipe 52. At this time, a contact width (contact area) in which the first arm portion 92 is in contact with the outer pipe 53 is larger than a contact width (contact area) in which the second arm portion 93 (protrusion portion 93a) is in contact with the movable pipe 52 (see FIGS. 9A and 9B described later). Thus, a contact load of the first arm portion 92 to the outer pipe 53 is larger than a contact load of the second arm portion 93 to the movable pipe 52. When the above-described contact load relationship is satisfied in the airtight member 9, sliding friction between the airtight member 9 (second arm portion 93) and the movable pipe 52 becomes small compared to a case where the first arm portion 92 slides on the movable pipe 52. The contact width referred to here corresponds to a contact length in a direction of the central axis Ax.

FIG. 9A is a cross-sectional view illustrating a part of the configuration of the airtight member included in the treatment tool according to the one embodiment of the disclosure. When the movable pipe 52 moves in the direction of the central axis Ax (direction of an arrow Y₁ in FIG. 9A) by operation of the operation knob 41, the movable pipe 52 slides with respect to the airtight member 9. At this time, the second arm portion 93 is deformed toward a side of the first arm portion 92 (direction of an arrow Y₂ in FIG. 9A) along with the movement of the movable pipe 52, whereby a contact area is kept constant. Note that when the second arm portion 93 is deformed to the side of the first arm portion 92, the movable pipe 52 and the airtight member 9 are in contact with each other and an airtight state is kept.

Furthermore, when the ultrasound treatment tool 2 is inserted into an abdominal cavity, abdominal air pressure is applied to the airtight member 9 from a distal end of the sheath 5 through a gap between the movable pipe 52 and the outer pipe 53. FIG. 9B is a cross-sectional view illustrating a part of the configuration of the airtight member included in the treatment tool according to the one embodiment of the disclosure. In the airtight member 9, abdominal air pressure P is applied to the first arm portion 92 and the second arm portion 93. When the abdominal air pressure P is applied to the first arm portion 92 and the second arm portion 93, the first arm portion 92 and the second arm portion 93 are deformed in directions of becoming away from each other (direction of arrows Y₃ and Y₄ in FIG. 9B). That is, the first arm portion 92 and the second arm portion 93 are deformed in directions in which a space formed between the first arm portion 92 and the second arm portion 93 expands. When the space formed between the first arm portion 92 and the second arm portion 93 expands, each of a load applied by the first arm portion 92 to the outer pipe 53 and a load applied by the second arm portion 93 to the movable pipe 52 is increased. Thus, airtightness can be kept further securely.

FIG. 10 is a cross-sectional view illustrating a part of the configuration of the airtight member included in the treatment tool according to the one embodiment of the disclosure. The cross-sectional view of FIG. 10 corresponds to the cross-sectional view of FIG. 8. In a cross section of the airtight member 9, when separation is performed by a line segment Q₁ that passes through a boundary between the first arm portion 92 and the second arm portion 93 and that is parallel to the central axis Ax9, and when it is assumed that a length between this line segment Q₁ and a straight line Q₂ passing through the outermost periphery of the first arm portion 92 is t₁, and a length between the line segment Q₁ and a straight line Q₃ passing through the innermost periphery of the second arm portion 93 (protrusion portion 93a) is t₂, a relationship of t₁<t₂ is preferably satisfied in order to reduce sliding friction and to secure airtightness.

Next, an example of an operation of the above-described treatment device 1 will be described. An operator holds the ultrasound treatment tool 2 in a hand and inserts a distal end portion of the ultrasound treatment tool 2 into an abdominal cavity through an abdominal wall, for example, by using a trocar or the like. Then, the operator grips a target region with the jaw 6 and the treatment unit 811 by operating the operation knob 41 and opening/closing the jaw 6 with respect to the treatment unit 811. Subsequently, the operator presses the operation button 42. Then, the energy controller 33 executes control described in the following.

The energy controller 33 controls an operation of the high-frequency current supplying unit 32, and supplies a high-frequency current between the jaw 6 and the ultrasound probe 8 through the pair of high-frequency lead wires C₂ and C₂′, the first conductive portion 711, the second conductive portion, and the horn 73. Also, substantially at the same time as the supply of the high-frequency current between the jaw 6 and the ultrasound probe 8, the energy controller 33 generates an ultrasound vibration in the ultrasound transducer 72 by controlling an operation of the ultrasound current supplying unit 31 and supplying AC power to the ultrasound transducer 72 through the pair of transducer lead wires C₁ and C₁′. That is, Joule heat is generated in the target region by flowing of a high-frequency current. Also, frictional heat is generated between a treatment surface and the target region due to a longitudinal vibration of the treatment unit 81. Then, the target region is incised while being coagulated.

In the embodiment described above, a gap between the outer pipe 53 and the movable pipe 52 is sealed by the airtight member 9 in the configuration including the movable pipe 52 that moves with respect to the outer pipe 53. This airtight member 9 secures airtightness between the outer pipe 53 and the movable pipe 52 by the first arm portion 92 and the second arm portion 93 that are bifurcated from the main body portion 91. Furthermore, when the movable pipe 52 moves, the second arm portion 93 is deformed to the side of the first arm portion 92. Thus, it is possible to reduce sliding friction and to control a variation in the sliding friction while keeping an airtight state between the movable pipe 52 and the airtight member 9. According to the present embodiment, it is possible to improve resistance of the airtight member 9 to the sliding of the movable pipe 52 by reducing the sliding friction and controlling the variation.

Furthermore, in the above-described embodiment, a configuration in which the protrusion portion 93a is brought into contact with the movable pipe 52 is included. Thus, a contact width (contact area) that generates the sliding friction can be made constant. Also, since the cross section of the airtight member 9 (see FIG. 8) is Y-shaped, sealing performance can be secured even in a state in which abdominal air pressure is applied.

Note that in the above-described embodiment, the description has been made on the assumption that the protrusion portion 93a is formed on the second arm portion 93 of the airtight member 9. However, a configuration that does not include a protrusion portion 93a may be used as long as a contact area with respect to an outer pipe 53 is larger than a contact area with respect to a movable pipe 52 when an airtight member 9 comes into contact with the outer pipe 53 and the movable pipe 52.

Also, in the above-described embodiment, the configuration including the movable pipe 52 and the outer pipe 53 has been described as an example. However, a member on an inner side (corresponding to a movable pipe 52) may be a solid member such as a rod member.

Also, in the above-described embodiment, the configuration in which the movable pipe 52 is inserted into the inside of the outer pipe 53 and the inner member is movable has been described as an example. However, an outer member (outer pipe 53) may be a movable member. In this case, a fixation member is provided inside the outer pipe 53 and is a pipe-shaped or solid member. Furthermore, in an airtight member provided between the members, a contact area on a side of the movable member is smaller than a contact area on a side of the fixation member.

### Modification Example

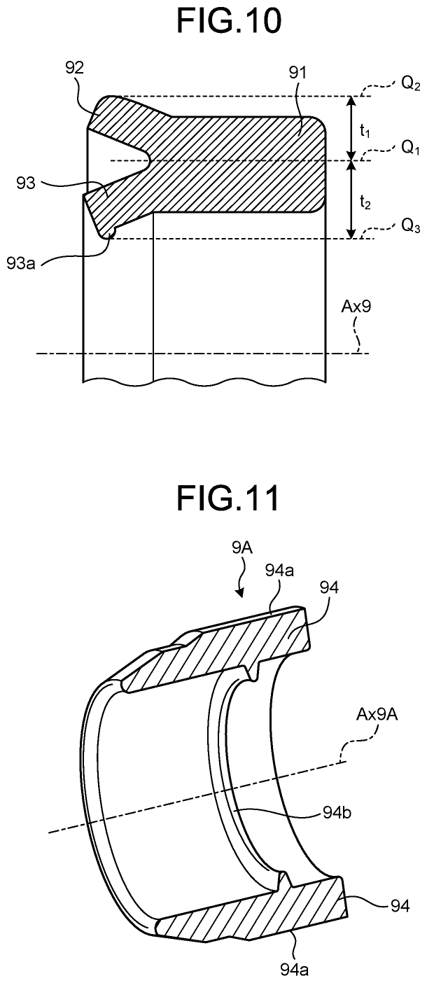

FIG. 11 is a cross-sectional view illustrating a part of a configuration of an airtight member included in a treatment tool according to a modification example of an embodiment of the disclosure. Similarly to the airtight member 9 described above, an airtight member 9A is in contact with an outer pipe 53 on an outer peripheral side and in contact with a movable pipe 52 on an inner peripheral side.

The airtight member 9A has a cylindrical main body portion 94. In the main body portion 94, a contact surface 94a that is provided on an outer peripheral surface and that is in contact with an inner peripheral surface of the outer pipe 53 is formed. The contact surface 94a is placed on the outermost periphery of the airtight member 9A in a natural state. The airtight member 9A is formed of an elastically deformable material such as rubber or resin. A central axis Ax9A corresponds to a central axis of a cylindrical shape of the main body portion 94.

The main body portion 94 includes a protrusion portion 94b that is provided on an inner peripheral surface and that protrudes toward the central axis Ax9A. A distal end of the protrusion portion 94b is placed in the innermost periphery in the airtight member 9A in the natural state.

When the airtight member 9A is attached to the movable pipe 52 and the outer pipe 53, the contact surface 94a is pressed against an inner peripheral surface of the outer pipe 53, and the protrusion portion 94b is pressed against an outer peripheral surface of the movable pipe 52. At this time, a contact area in which the contact surface 94a is in contact with the outer pipe 53 is larger than a contact area in which the protrusion portion 94b is in contact with the movable pipe 52.

When the movable pipe 52 moves in a direction of the central axis Ax9A by operation of an operation knob 41, the movable pipe 52 slides with respect to the airtight member 9A. At this time, since the protrusion portion 94b is deformed toward a side of the moving direction, it is possible to reduce sliding friction while keeping an airtight state between the movable pipe 52 and the airtight member 9A.

Although embodiments of the disclosure have been described above, the disclosure is not limited only by the above-described embodiments. The disclosure may also include various embodiments that are not described herein. In the above-described embodiments, a treatment device 1 is configured to apply an ultrasound vibration or high-frequency current to a body tissue. However, this is not a limitation, and a configuration to apply only one of an ultrasound vibration and high-frequency current may be employed, a configuration to apply thermal energy may employed, or a configuration that can selectively apply an ultrasound vibration, high-frequency current, and thermal energy may be employed. Also, the above-described airtight member may be employed in a configuration in which a configuration of applying energy such as an ultrasound vibration is not included and a body tissue is only gripped.

Also, in the above-described embodiments, the description has been made on the assumption that the airtight member 9 is provided on a proximal end side of the outer pipe 53, but an airtight member 9 may be provided at a center or on a distal end side of an outer pipe 53.

According to the disclosure, there is an effect that it is possible to control sliding friction while securing airtightness between members when one member moves with respect to another member.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A treatment tool comprising:
a handle;
an elongated fixation member, a proximal end side of the elongated fixation member being fixed to the handle;
a movable member that is provided coaxially with the fixation member, the movable member being configured to move relative to the fixation member; and
an airtight member provided between the fixation member and the movable member, the airtight member having a first contact width that contacts the fixation member and a second contact width that contacts the movable member, the second contact width being smaller than the first contact width.

2. The treatment tool according to claim 1, wherein
the airtight member includes:
a cylindrical main body portion,
a first arm portion that extends along a first incline in a direction away from the central axis of the main body; and
a second arm portion that extends along a second incline in a direction towards the central axis,
the first arm portion contacts the fixation member, and
the second arm portion contacts the movable member.

3. The treatment tool according to claim 2, wherein
the second arm portion includes a protrusion portion on an outer peripheral side, and
the protrusion portion contacts the movable member.

4. The treatment tool according to claim 1, wherein
each of the fixation member and the movable member has a pipe shape.

5. The treatment tool according to claim 4, wherein
the movable member is inserted into an inside of the fixation member.

6. The treatment tool according to claim 4, further comprising
a gripping member configured to be inserted into the movable member, the gripping member being configured to protrude from a distal end of the movable member, and
a jaw that is attached to a distal end side of the fixation member and a distal end side of the movable member, the jaw being configured to grip a body tissue with the gripping member and rotate about a predetermined central axis, wherein
the jaw is configured to rotate in conjunction with movement of the movable member.

7. The treatment tool according to claim 1, wherein
the handle has an operation knob configured to rotate with respect to the handle, and
the movable member is configured to move relative to the fixation member in conjunction with the rotation of the operation knob.

8. The treatment tool according to claim 6, further comprising
an ultrasound transducer configured to generate an ultrasound vibration, wherein
the gripping member is configured to vibrate in a longitudinal direction due to the ultrasound vibration generated by the ultrasound transducer.

9. The treatment tool according to claim 6, wherein
a high-frequency current flows in the gripping member, and
the gripping member and the jaw form a pair of electrodes to conduct the high-frequency current.

10. The treatment tool according to claim 2, wherein
the airtight member is configured to be deformed in a direction in which the first arm portion and the second arm portion are separated from each other when abdominal air pressure is applied to the first arm portion and the second arm portion.

11. The treatment tool according to claim 2, wherein
a first contact area in which the fixation member contacts the first arm portion is larger than a second contact area in which the movable member contacts the second arm portion.

12. The treatment tool according to claim 2, wherein
when separation is performed by a line segment that passes through a boundary between the first arm portion and the second arm portion and that is parallel to the central axis, and when a length between the line segment and an outer periphery of the first arm portion is t₁ and a length between the line segment and an outer periphery of the second arm portion is t₂, a relationship of t₁<t₂ is satisfied.

13. The treatment tool according to claim 2, wherein
a contact load between the first arm portion and the fixation member is larger than a contact load between the second arm portion and the movable member.

14. A treatment tool airtight member that is provided between a fixation member and a movable member, the treatment tool airtight member being configured to create an airtight seal around the fixation member and the movable member, a proximal end side of the fixation member being fixed to a handle, the movable member being provided coaxially with the fixation member and moving relative to the fixation member, wherein
the airtight member includes a second contact width that contacts the movable member and a first contact width that contacts the fixation member, the second contact width being smaller than the first contact width.

15. The treatment tool airtight member according to claim 14, comprising
a cylindrical main body portion,
a first arm portion extending from along a first incline in a direction of becoming away from the central axis, and
a second arm portion extending f along a second incline in a direction towards the central axis, wherein
the first arm portion contacts the fixation member, and
the second arm portion contacts the movable member.
